(19) United States
(12) Patent Application Publication
(10) Pub. No.: US 2013/0052183 A1
(43) Pub. Date: Feb. 28, 2013

(54) METHODS FOR THE TREATMENT OF MORGELLONS DISEASE

(71) Applicant: Gordon Stamp, Excelsior, MN (US)

(72) Inventor: Gordon Stamp, Excelsior, MN (US)

(73) Assignee: Gordon Stamp. R.N., Excelsior, MN (US)

(21) Appl. No.: 13/593,147

(22) Filed: Aug. 23, 2012

Related U.S. Application Data
(60) Provisional application No. 61/528,042, filed on Aug. 26, 2011.

(51) Int. Cl. A61K 38/47; A61P 17/00

(52) U.S. Cl. 424/94.61

(57) ABSTRACT

Provided herein are compositions and methods of using such compositions to treat Morgellons and other symptoms associated with skin and nail diseases and disorders.

### CROSS-REFERENCE TO RELATED APPLICATIONS

This application claims the benefit of U.S. Provisional Application No. 61/528,042, filed Aug. 26, 2011, entitled “METHODS FOR THE TREATMENT OF MORGELLONS DISEASE,” which is incorporated herein by reference in its entirety.

### BACKGROUND

Morgellons (also known as Morgellons disease or Morgellons syndrome) is a condition characterized by a range of various dermatologic and neuropsychiatric symptoms. These symptoms include itching, crawling, biting, and stinging sensations; presence of fibers on, within or protruding from the skin (can be black, white, blue, red or combinations thereof); persistent skin lesions (e.g., rashes, sores, pimple-like pustules); presence of black specks and/or granules that resemble salt or sand; and presence of insects on the skin or within an open lesion. Morgellons is also characterized by joint arthralgia, fatigue, sleep disturbances, changes in vision, mental confusion and memory loss. Morgellons can cause extreme discomfort; in severe cases, it can lead to anxiety, depression and suicide.

The etiology of Morgellons is highly controversial. The scientific and medical communities have yet to confirm a causative agent. It is currently unknown whether and which infectious agents cause Morgellons, in particular because the disease resembles and is often confused with delusional parasitosis. The Centers for Disease Control and Prevention (CDC) states that it is not known at present whether the condition represents a new disease entity, or whether persons who identify themselves as having Morgellons have a common cause for their symptoms, share common risk factors, or are contagious. Various agents, including bacteria, viruses, fungi, certain plant species, insect larvae, and other parasitic organisms including nematodes have been proposed to cause Morgellons and the disease has been associated with Lyme disease. However, only in some cases does the disease respond to antibacterial therapy (Savely et al., 2006).

### SUMMARY

The present invention provides methods and compositions for disease treatment, especially for treatment of Morgellons. It has been discovered that compositions containing cellulase are effective in relieving, reducing, inhibiting or ameliorating symptoms associated with skin and nail conditions. In preferred embodiments, the condition is Morgellons. The compositions of the invention may be administered topically and/or systemically. Systemic administration of the compositions of the invention may also be accomplished through transdermal administration.

In accordance with one aspect of the invention, a topical or dermatological composition for treatment of Morgellons is provided. In some embodiments, the topical composition consists essentially of a treatment-effective amount of cellulase and a dermatologically acceptable carrier. The compositions described herein provide an effective treatment for Morgellons upon topical application to skin in need thereof.

The compositions described herein are contemplated to include up to about 75% cellulase, up to about 50% cellulase, up to about 25% cellulase, up to about 10% cellulase, or up to about 5% cellulase. In some embodiments, the compositions include about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29% or about 30% cellulase. In preferred embodiments, the compositions include about 3% to about 10% cellulase. In further preferred embodiments, the compositions include about 4% to about 7% cellulase.

In accordance with another aspect of the invention, a method for treating Morgellons in a patient is provided. The method includes topically applying a dermatological composition containing an effective amount of cellulase to the patient. In some embodiments, the dermatological composition further includes an additive including, but not limited to, a preservative, an antioxidant, a fragrance, a skin penetration enhancer, a colorant, a sunscreen, or combinations thereof.

In some embodiments, the composition is a cream. In other embodiments, the composition is a lotion. In other embodiments, the composition is a solution. In still further embodiments, the composition is an ointment. In still further embodiments, the composition is a spray.

Also provided is a kit including the cellulase of the invention including instructional material for use.

In some embodiments, the compounds and methods of the invention are used to treat additional or distinct symptoms associated with skin disorders. Additional or distinct symptoms of pathogenic infections and parasitic infestations include sensations of biting, crawling and stinging on and under the skin; presence of skin fibers on, within or protruding from the skin (can be black, white, blue and/or red); presence of worm-like organisms within the skin or expelled from the bowel or bladder; secretions from the eyes, nasal passages, vagina and oral cavity; pimple-like pustules or rashes; presence of black specks and/or granules that resemble salt or sand; presence of insects on the skin or within an open lesion; chronic fatigue; memory loss; difficulty concentrating; mood swings; depression; joint swelling; fibromyalgia; visual decline; reduced immunity to common illnesses; hair loss; and sleep disorders. The lesions and other symptoms do not generally heal under conventional medical intervention. Antibiotic, antifungal and antiparasitic treatments have proven largely to be of no benefit.

In further embodiments, the compounds and methods of the invention are used to treat symptoms associated with nail diseases including, but not limited to, onychomycosis (fungal infection of the nail) and Morgellons symptoms that mimic onychomycosis.

In still further embodiments, the compounds and methods of the invention are used to treat symptoms associated with athlete's foot (tinea pedis) and Morgellons symptoms that mimic athlete's foot.

In still further embodiments, the compounds and methods of the invention are used to treat symptoms associated with skin disorders.

### BRIEF DESCRIPTION OF THE FIGURES

FIG. 1: (A) a Morgellons patient bad been suffering from a large, deep lesion on the back for several months. The lesion failed to heal using traditional antibiotic therapies. (B) a solution of cellulase was applied topically and significant improvements were observed within hours of application.

FIG. 2: A Morgellons sufferer had a large, open lesion on the skin. The patient applied a solution of cellulase topically once or twice daily for 4 months. (A) the lesion before initiating treatment; (B) the lesion four months after initiating treatment.

FIG. 3: Treatment of toenail fungus. (A) and (B) toenail fungus before treatment; (C) immersing a foot in cellulase solution; (D) a foot after cellulase treatment.

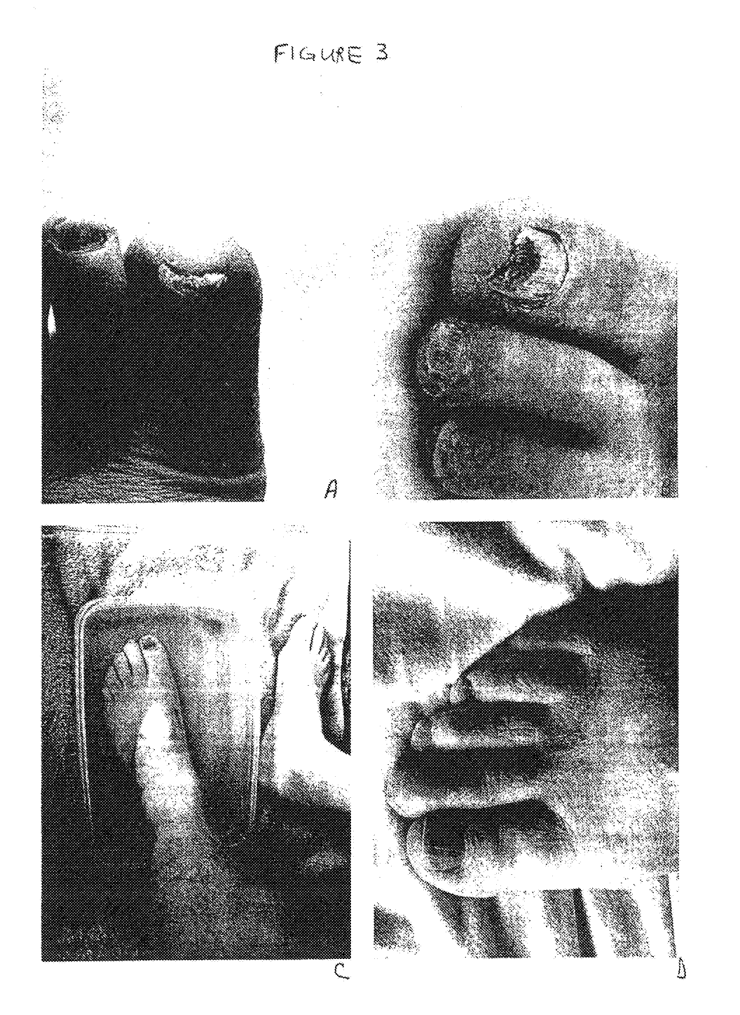

### DETAILED DESCRIPTION

### Definitions

As used herein, the terms “a” or “an” are used, as is common in patent documents, to include one or more than one, independent of any other instances or usages of “at least one” or “one or more.”

As used herein, the term “and/or” is meant to be both inclusive and exclusive, such that “A and/or B” includes “A but not B,” “B but not A,” and “A and B,” unless otherwise indicated.

As used herein, the term “about” is used to refer to an amount that is approximately, nearly, almost, or in the vicinity of being equal to a stated amount. The terms “about” and “approximately” are used interchangeably throughout this document.

The term “aerosol” refers to a suspension of fine solid particles or liquid droplets in a gas. The term “aerosol foam” refers to substance that is formed by trapping many gas bubbles in a liquid or solid. Cellulase can be incorporated into a foam material for delivery onto an affected surface incorporated into a foam. The term “aerosol metered” refers to a device that helps deliver a specific amount of a medication (such as cellulase) by supplying a short burst of aerosolized medicine. The term “aerosol powder” refers to a type of dispensing system which creates an aerosol mist of solid particles. The term “aerosol spray” refers to a type of dispensing system which creates an aerosol mist of liquid particles.

As used herein, the phrase “consisting essentially of” limits a composition to the specified materials or steps and those additional, undefined components that do not materially affect the basic and novel characteristic(s) of the composition.

The term “cream” refers to topical preparations for application to the skin or mucous membranes such as those of the rectum or vagina. Creams are semi-solid emulsions that are mixtures of oil and water. They are divided into two types: oil-in-water (O/W) creams that are composed of small droplets of oil dispersed in a continuous aqueous phase, and water-in-oil (W/O) creams that are composed of small droplets of water dispersed in a continuous oily phase.

The term “emulsion” refers to a mixture of two or more immiscible (unblendable) liquids. One liquid (the dispersed phase) is dispersed in the other (the continuous phase). Emulsions can be oil-in-water emulsions or water-in-oil emulsions.

The term “gel” or “jelly” refers to solid, jelly-like materials made up of a substantially dilute crosslinked system, which exhibits no flow when in the steady-state. By weight, gels are mostly liquid, yet they behave like solids due to a three-dimensional crosslinked network within the liquid.

The term, “gelling agent” refers to materials used to thicken and stabilize liquid solutions, emulsions, and suspensions. They dissolve in the liquid phase as a colloid mixture that forms an internal structure giving the resulting gel an appearance of a solid matter, while being mostly composed of a liquid. Gelling agents are very similar to thickeners.

As used herein, “instructional material” includes a publication, a recording, a diagram, or any other medium of expression which can be used to communicate the usefulness of the compositions of the invention in the kit for effecting alleviation, inhibition, amelioration, reduction or relief of the various diseases or disorders recited herein. Optionally, or alternately, the instructional material may describe one or more methods of alleviation the diseases or disorders in a cell or a tissue of a subject. The instructional material of the kit of the invention may, for example, be affixed to a container which contains the cellulase of the invention or be shipped together with a container which contains the cellulase. Alternatively, the instructional material may be shipped separately from the container with the intention that the instructional material and the compound be used cooperatively by the recipient.

The term “isolated” refers to a compound or compounds which is or are not associated with one or more proteins or one or more cellular components that are associated with the compound in vivo.

The term “lotion” refers to a low- to medium-viscosity topical preparation.

The term “nail disease” or “nail condition” refers to any infection, deformation, thickening, discoloration or inflammation of the fingernail or toenail. Onychomycosis (also known as “dermatophytic onychomycosis,” “ringworm of the nail” and “tinea unguium”) is the number one diagnosed and treated disease by podiatrists today. It is present in 2 to as high as 5 percent of the population. It has been reported that as much as 70 percent of the population has fungus recovered from the feet. The causative pathogens of onychomycosis include dermatophytes, Candida, and nondermatophytic molds, in particular members of the mold generation Scytalidium (name recently changed to Neoscytalidium), Scopulariopsis, and Aspergillus. The pathogens attack the nail, thriving off keratin, the nail's protein substance. When the tiny organisms take hold, the nail may become thicker, yellowish-brown or darker in color, and foul smelling. Debris may collect beneath the nail plate, white marks frequently appear on the nail plate, and the infection is capable of spreading to other toenails, the skin, or the fingernails. As described herein, nail disease can accompany Morgellons and/or Morgellons symptoms.

The term “ointment” refers to a viscous, homogeneous, semi-solid preparation used topically on a variety of body surfaces, such as the skin and the mucus membranes of the eye (an eye ointment), vagina, anus, and nose.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like, In particular, parenteral administration is contemplated to include, but is not limited to, subcutaneous, intraperitoneal, intramuscular, intrasternal injection, and kidney dialytic infusion techniques.

The term “paste” refers to a pharmacological form consisting of a fatty base, water, and at least a solid substance in which a solid particulate is suspended.

The phrase “pharmaceutically acceptable” means a carrier, vehicle, diluent, excipient, and/or salt that is compatible with the other ingredients of the formulation, and not deleterious to the recipient thereof. Suitable pharmaceutically acceptable carriers or vehicles include polymeric matrices, sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, silicone oils, humectants, moisturizers, viscosity modifiers, emulsifiers and stabilizers. The compositions may also contain auxiliary substances, i.e. antimicrobial agents such as chlorobutanol, parabens or organic mercurial compounds; and pH adjusting agents such as sodium hydroxide, hydrochloric acid or sulfuric acid. The final composition should be sterile, essentially free of foreign particles, and have a pH that allows for optimum drug stability.

“Skin diseases” or “skin disorders” are defined as any disease or disorder of the skin or mucus membranes, including, but not limited to, Morgellons, acne (e.g., Propionibacterium acnes), athlete's foot, canker sores, carbuncles, candidiasis (including, but not limited to, oral, vaginal, penile, diaper area (diaper rash) and in skin folds (candidal intertrigo)), bacterial vaginitis, vaginosis (bacterial, protozoan, fungal including candida albicans; Gardnerella vaginalis; Trichomonas vaginalis), cellulitis, cold sores, dandruff, dermatitis (including, but not limited to, atopic dermatitis, contact dermatitis, serborrhoeic dermatitis, cradle cap, nummular dermatitis, perioral dermatitis, and dermatitis herpetiformis), eczema, erythrasma, erysipelas, erythema multiforme, furuncles, folliculitis, impetigo, infection (including, but not limited to, streptococcal infections), lice infection (head lice, body lice, crab/pubic lice), fungal infections. Ritter's disease, dermatophytosis (caused by Tinea pedis, Tinea unguium, Tinea corporis, Tinea cruris, Tinea manuum, Tinea capitis, Tinea barbae, Tinea faciei and Tinea versicolor), protozoal infection, Trichomycosis or vesicular bullous eruptions, and infections and infestations from flies (black flies, no-see-ums, sandflies, snipe flies, horse flies, deer flies, stable flies, human bot flies, screw worms), fleas, bedbugs, mites (including scabies), ticks, Collembola, and Strepsiptera.

Athlete's foot (also known as ringworm of the foot and tinea pedis) is a fungal infection of the skin that causes scaling, flaking, and itch of affected areas. It is caused by fungi in the genus Trichophyton and is typically transmitted in moist areas where people walk barefoot, such as showers or bathhouses. Although the condition typically affects the feet, it can spread to other areas of the body, including the groin. Athlete's foot causes sealing, flaking, and itching of the affected skin. Blisters and cracked skin may also occur, leading to exposed raw tissue, pain, swelling, and inflammation. Secondary bacterial infection can accompany the fungal infection. If the fungal infection spreads to other areas of the body, such as the groin, it is usually is called by a different name, such as tinea corporis on the body or limbs and tinea cruris (jock itch or dhobi itch) for an infection of the groin.

In certain embodiments, the compositions and methods are used in the treatment of additional symptoms associated with other pathological conditions of the skin (skin diseases or skin disorders), described elsewhere herein and including but not limited to urticaria (hives), varicella (chicken pox), psoriasis, eczema, sunburn, athlete's foot, hydradenitis suppurativa, dry skin (xerosis), perforating dermatoses (Kyrle's disease, perforating folliculitis, reactive perforating collagenosis, elastosis perforans serpiginosa, acquired perforating dermatosis), photodermatitis, parasitic infestations including but not limited to scabies, lice (head, body, pubic) and filariasis, insect bites, including but not limited to those from mosquitoes or chiggers, allergic reaction to contact with specific chemicals, such as Urushiol, derived from Poison Ivy or Poison Oak, shaving, dandruff, pigmented moles, punctate palmoplantar keratoderma, internal conditions including but not limited to kidney failure (uraemia), liver diseases including but not limited to hepatitis, thyroid disease including both hyper- and hypo-thyroid hormone levels, blood disorders including but not limited to lymphomas, iron deficiency anemia, diabetes mellitus, jaundice, polycythemia vera, cholestasis, multiple myeloma, Hodgkin's disease, neurologic conditions including but not limited to pinched nerves and post herpetic neuralgia, and infectious diseases including but not limited to Lyme disease and HIV. In other embodiments, the compounds of the invention are used to treat additional symptoms associated with the pathological conditions described herein.

The term “solution” refers to a system at chemical equilibrium in which a solute (liquid, solid, or gas) is dissolved in a liquid solvent. In some embodiments, the invention includes application of a solution of cellulase to the skin. In preferred embodiments, the cellulase is present in a solid particulate form before being dissolved in a solvent. In preferred embodiments, the solvent is water. Solid particulate solutes contemplated for use in the instant invention include a range of particle sizes, from granular to fine to micronized.

The term “spray” refers to a collection of liquid drops and the entrained surrounding gas. The term “spray metered” refers to a device that helps deliver a specific amount of a medication (such as cellulase) by supplying a short burst of liquid drops and the entrained surrounding gas. The term “spray suspension” refers to a suspension of an active agent (such as cellulase) in a liquid such that it can be sprayed onto a surface (such as skin) as a suspension of the active agent in a very small drops of liquid entrained in surrounding gas.

The terms “subject” and “patient” as used herein include mammals, such as humans. Mammals also include, but are not limited to, farm animals, sport animals and pets. Pets include, but are not limited to, dogs and cats.

A “surfactant” or “surface-active agent” refers to an organic compound that reduces the surface tension when dissolved in water or water solutions. In an emulsion, a surfactant will contain a hydrophilic portion and a lipophilic portion by which it functions to reduce the surface tension of the surfaces between immiscible phases. Functionally, in dermatological applications, surfactants include emulsifying agents, wetting agents, cleansing agents, foam boosters, and solubilizing agents. A surfactant is any nonionic, anionic, cationic or zwitterionic (e.g., including, but not limited, betaines (e.g., cocamidopropyl betaine), detergents and amino acids) compound of moderate to high molecular weight (such as from about 100 to 300,000 Daltons) for which a significant portion of the molecule is hydrophilic and a significant portion is lipophilic.

The term “suspension” refers to a mixture in which fine particles are suspended in a fluid where they are supported by buoyancy; as well as a mixture in which fine particles are more dense than the fluid and are not supported by buoyancy.

“Therapeutically effective amount” or “effective amount” is intended to include an amount of a compound described herein, or an amount of a combination of compounds described herein, to treat or prevent a pathologic condition (disease or disorder), or to treat at least one symptom of the pathologic condition (disease or disorder) in, for example, a mammal. The combination of compounds can be a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. A synergistic effect can be demonstrated at suboptimal concentrations of the compounds. Synergy can be in terms of lower cytotoxicity, increased activity, or some other beneficial effect of the combination compared with the individual components. In some embodiments, the cellulase of the invention is applied or administered in combination with other compounds, such as anti-infectives, anti-parasitics, anti-virals, anti-fungals and antibacterials.

The term “topical” refers to any surface of a body tissue or organ. A topical formulation is one that is applied to a body surface, such as an eye, to treat that surface or organ. Topical formulations include liquid drops such as eye drops; creams, lotions, sprays, emulsions, and gels.

As used herein, “treating” or “treat” includes (i) preventing a pathologic condition (e.g., a disease or disorder) from occurring (e.g. prophylaxis); (ii) inhibiting the pathologic condition or arresting its development; (iii) relieving the pathologic condition; and/or (iv) diminishing at least one symptom associated with the pathologic condition. In certain embodiments of the invention described herein, “treating” can include improvement in Morgellons symptoms. “Treating” is also intended to include improvement in skin appearance, improvement in skin condition, reduction in itching, reduction in crawling, stinging or biting sensations on or under the skin, reduction in inflammation and reduction of skin lesions, regardless of the cause. Morgellons has been associated with and may be a causative agent in systemic symptoms including joint arthralgia, fatigue, sleep disturbances, changes in vision, mental confusion and memory loss. In some embodiments, the compositions and methods described herein may treat systemic Morgellons symptoms as well as skin and nail symptoms.

References in the specification to “one embodiment,” “an embodiment,” “an example embodiment, ” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Reference will now be made in detail to certain embodiments of the disclosed subject matter, examples of which are illustrated in the accompanying descriptions and formulas. While the disclosed subject matter will be described in conjunction with the enumerated claims, it will be understood that they are not intended to limit the disclosed subject matter to those claims. On the contrary, the disclosed subject matter is intended to cover all alternatives, modifications and equivalents, which may be included within the scope of the presently disclosed subject matter as defined by the claims.

Cellulase refers to a group of enzymes produced primarily by fungi, bacteria, and protozoans which catalyze the hydrolysis of cellulose, the principal constituent of the cell wall of plants. As used herein, the term “cellulase” may refer to a single enzyme (momopreparation) having the ability to hydrolyze cellulose. The term “cellulase” may also refer to a multienzymatic preparation or complex containing two or more individual enzymes that play essential roles in the hydrolysis of cellulose to glucose. In some embodiments, the methods of the invention employ a cellulase that is a multienzymatic complex containing at least three physically and chemically distinct enzyme components that play essential roles in the hydrolysis of cellulose.

Commonly, three types of reactions are catalyzed by cellulases. First, the noncovalent interactions present in the crystalline structure of cellulose are broken (endocellulase activity). Second, the individual cellulose fibers are hydrolyzed to break it into smaller sugars (exocellulase activity). Third, disaccharides and tetrasaccharides are hydrolyzed into glucose (cellobiase/beta-glucosidase activity).

Cellulases contemplated for use in the methods of the instant invention include, but are not limited to, endocellulases, exocellulases, cellobiases, beta-glucosidases, oxidative cellulases, and cellulose phosphorylases. Endocellulases (including, but not limited to endoglucanases) break internal bonds to disrupt the crystalline structure of cellulose and expose individual cellulose polysaccharide chains. Exocellulases (including, but not limited to exoglucanases) cleave two to four units from the ends of the exposed chains produced by endocellulase, resulting in the tetrasaccharides or disaccharides, such as cellobiose. There are two main types of exocellulases (or cellobiohydrolases): one type works processively from the reducing end, and one type works processively from the nonreducing end of cellulose. Cellobiases or beta-glucosidases hydrolyze the exocellulase product into individual monosaccharides. Oxidative cellulases depolymerize cellulose by radical reactions, for instance cellobiose dehydrogenase (acceptor). Cellulose phosphorylases depolymerize cellulose using phosphates instead of water.

The cellulase contemplated for use in the invention may include any enzyme involved in the degradation of cellulose to glucose, xylose, mannose, galactose, or arabinose and may have activity in the acid, neutral, and/or alkaline pH range. The cellulase may be of fungal or bacterial origin, which may be obtainable or isolated and purified from microorganisms which are known to be capable of producing cellulases, e.g., species of Humicola, Coprinus, Thielavia, Fusarium, Myceliophthora, Acremonium, Cephalosporium, Scytalidium, Penicillium or Aspergillus (see, for example, EP 458162), especially those produced by a strain selected from the species Humicola insolens (reclassified as Scytalidium thermophilum, see for example, U.S. Pat. No. 4,435,307), Coprinus cinereus, Fusarium oxysporum, Myceliophthora thermophila, Meripilus giganteus, Thielavia terrestris, Acremonium sp., Acremonium persicinum, Acremonium acremonium, Acremonium brachypenium, Acremonium dichromosporum, Acremonium obelavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obelavatum CBS 311.74, Acremonium pinkertoniae CBS 157.70, Acremonium roseogriseum CBS 134.56, Acremonium incoloratum CBS 146.62, and Acremonium furatum CBS 299.7011. Cellulases may also be obtained from Trichoderma (particularly Trichoderma viride, Trichoderma reesei, and Trichoderma koningii), alkalophilic Bacillus (see, for example, U.S. Pat. No. 3,844,890 and EP 458162), and Streptomyces (see, for example, EP 458162).

The cellulase used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, Calif., 1991). Suitable media are available from commercial suppliers or may be prepared according to published compositions (e.g., in catalogs of the American Type Culture Collection). Temperature ranges and other conditions suitable for growth and cellulase production are known in the art (see, e.g., Bailey. J. E., and Ollis, D. F., Biochemical Engineering Fundamentals, McGraw-Hill Book Company, N.Y., 1986).

The fermentation can be any method of cultivation of a cell resulting in the expression or isolation of a cellulase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the cellulase to be expressed or isolated.

The resulting cellulase produced by the methods described above may be recovered from the fermentation medium by conventional procedures including, but not limited to, centrifugation, filtration, spray-drying, evaporation, or precipitation. The recovered enzyme may then be further purified by a variety of chromatographic procedures, e.g., ion exchange chromatography, gel filtration chromatography, affinity chromatography, or the like.

Examples of cellulase suitable for use in the present invention include, but are not limited to, glucose oxidase, xylanase, hemicellulase, amylase (fungal and bacterial) (available from Enzymology Research Center, Inc., Miltona, Minn.), cellulase (available from Columbus Chemical Industries, Columbus, Wis.), CELLUCLAST™ (available from Novozymes A/S) and NOVOZYM™ 188 (available from Novozymes A/S). Other commercially available preparations comprising cellulase which may be used include CELLUZYME™, CEREFLO™ and ULTRAFLO™ (Novozymes A/S), LAMINEX™ and SPEZYME™ CP (Genencor Int.), and ROHAMENT™ 7069 W (Rohm GmbH).

The cellulase enzymes may be added in amounts from about 0.001% to about 30.0% wt. of solids, mote preferably from about 1.0% to about 20.0% wt, of solids, and most preferably from about 3.0% to about 8,0% wt. of solids.

As mentioned above, the cellulase used in the methods of the present invention may be a monocomponent preparation, i.e., a component essentially free of other cellulase components, or a multicomponent preparation, i.e., a preparation containing multiple cellulase enzyme components. A single cellulase component may be a recombinant component, i.e., produced by cloning of a DNA sequence encoding the single component and subsequent cell transformed with the DNA sequence and expressed in a host (see, for example, WO 91/17243 and WO 91/17244). Other examples of monocomponent cellulases include, but are not limited to, those disclosed in JP-07203960-A and WO-9206209. The host is preferably a heterologous host (enzyme is foreign to host), but the host may under certain, conditions also be a homologous host (enzyme is native to host). Monocomponent cellulases may also be prepared by purifying such an enzyme from a fermentation medium.

Cellulase amounts are measured by the activity that is catalyzed by the enzyme. Cellulase activities contemplated for use in the methods described herein range from about 1,000 CU/g to about 150,000 CU/g. In preferred embodiments, the cellulase activity is at least about 1,000 CU/g. In other preferred embodiments, the cellulase activity ranges from about 1,000 to about 20,000 CU/g. In more preferred embodiments, the cellulose activity ranges from about 2,500 to about 10,000 CU/g. In the most preferred embodiments, the cellulase activity ranges from about 4,000 to about 8,000 CU/g.

The relationship between activity and concentration is affected by many factors such as temperature, pH and specific process steps. An enzyme assay must be designed so that the observed activity is proportional to the amount of enzyme present in order that the enzyme concentration is the only limiting factor. One way to determine cellulase activity, measured in terms of Cellulase Viscosity Unit (CEVU), quantifies the amount of catalytic activity present in a sample by measuring the ability of the sample to reduce the viscosity of a solution of carboxymethyl cellulose (CMC). The assay is carried out at 40 degrees Celsius in 0.1 M phosphate pH19.0 buffer for 30 minutes with CMC as substrate (33.3 g/L carboxymethyl cellulose Hercules 7 LFD) and an enzyme concentration of approximately 3.3-4.2 CEVU/ml. The CEVU activity is calculated relative to a declared enzyme standard, such as CELLUZYME™ Standard 17-1194 (obtained from Novozymes A/S, Bagsvaerd, Denmark). The reduction in viscosity may be determined by a vibration viscosimeter (e.g., MIVI 3000 from Sofraser, France).

In preferred embodiments, the cellulase has CAS Number 9012-54-8 and is provided as a solid particulate or powder. The cellulase is dissolved in water and applied topically.

In other embodiments, a muitienzymatic combination is employed. For example, a powdered composition containing 40% cellulase, 20% hemicellulase, 20% xylanase and 20% glucose oxidase is dissolved in water and applied topically. In some embodiments, the powdered composition containing cellulase, hemicellulase, xylanase and glucose oxidase is dissolved in water to a final concentration of about 1 to about 10%. In preferred embodiments, the powdered composition containing cellulase, hemicellulase, xylanase and glucose oxidase is dissolved in water to a final concentration of about 2% to about 8%.

### Formulations

The compounds (i.e., cellulase) described herein may be topically applied in pure form. However, it will generally be desirable to administer the compounds to the skin as a composition or formulation, in combination with a dermatologically acceptable carrier, which may be a solid or a liquid.

Useful solid carriers include finely divided solids such as talc, clay, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

In some embodiments, the compounds may be applied as a topical cream or lotion in which the compounds are dissolved or dispersed or both partially dissolved and partially dispersed. Topical creams or lotions may be either oil-in-water emulsions or water-in-oil emulsions. The oil phase may include but is not limited to fatty alcohols, acids, or esters such as cetyl palmitate, cetyl alcohol, stearyl alcohol, stearic acid, isopropyl stearate, glycerol stearate, mineral oil, white petrolatum, or other oils alone or in combination.

Emulsifiers that may be added to the composition include, but are not limited to, steareth 20, ceteth 20, sorbitan sesquioleate, sorbitan mono-oleate, propylene glycol stearate, dosium lauroyl sarcosinate, polysorbate 60, or combination. Preservatives, antioxidants, fragrances, colorants, sunscreens, thickeners, and other additives required to achieve pharmaceutical or cosmetically acceptable or preferred product may also be included. However, topical creams and lotions are not limited to these components since one skilled in the art will be aware of additional components useful in the formulation of topical creams and lotions.

In another embodiment the compound may be applied as a solution or suspension. These are fluid solvent or mixed-solvent systems including, but not limited to, water, ethanol, propylene glycol, glycerol, polyethylene glycol, ethyl acetate, propylene carbonate, n-methyl pyrolidone, triethanolamine, 1,4-butanediol, triacetin, diacetin, dimethyl isosorbide alone or in combination. Preservatives, antioxidants, fragrances, colorants, sunscreens, thickeners, suspending agents, enhancers, and other additives required to achieve pharmaceutically or cosmetically acceptable or preferred product may also be included. Again, topical solutions or suspensions are not limited to these components, since one skilled in the art will be aware of additional components useful in the formulation of topical solutions or suspensions. In some embodiments, the solutions of the invention should be used within 72 hours of initial mixing to ensure freshness.

The compound may also be applied using a pharmaceutical or cosmetic carrier form such as an ointment, roll-on or stick product, micro-emulsion, shake powder, an aerosolized spray or mousse, a pump spray or mousse, or bath additive. In some embodiments, the compound is applied topically using an applicator, sponge or nail brush. Examples of ointments include essentially non-aqueous mixtures of petrolatum, lanolin, polyethylene glycol plant or animal oils, either hydrogenated or otherwise chemically modified. An ointment may also contain a solvent in which cellulase is either fully or partially dissolved. Additional pharmaceutical carriers will be known to those skilled in the art and this list should not be considered to be limiting.

Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably, the gelling agent comprises between about 0.2% to about 4% by weight of the composition. More particularly, the preferred compositional weight percent range for “CARBOPOL®” is between about 0.5% to about 2%, while the preferred weight percent range for “NATROSOL®” and “KLUCEL®” is between about 0.5% to about 4%. The preferred compositional weight percent range for both “HYPAN®” and “STABILEZE®” is between about 0.5% to about 4%.

“CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. Other preferred gelling polymers include MVA/MA copolymers, MVE/MA decadiene crosspolymer, PVM/MA copolymer, or a combination thereof.

Preservatives may also be used in this dermatological composition and preferably comprise about 9.05% to 0.5% by weight of the total composition. The use of preservatives assures that if the product is microbially contaminated, the formulation, will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

One or more chelating agents may be employed in the dermatological composition described herein, to bind metal ions. Chelating agents may act as antioxidants, preservatives, and lubricating agents. A preferred chelating agent for use in the dermatological composition described herein is ethylenediaminetetraacetic acid (EDTA). EDTA may be provided as a free acid, a disodium salt, at 0.1M and a titrant of 0.01M (Columbus Chemical Industries. Columbus, Wis.).

Titanium dioxide may be used as a sunscreen to serve as prophylaxis against photosensitization. Alternative sunscreens include methyl cinnamate. Moreover, BHA may be used as an antioxidant, as well as to protect ethoxydiglycol and/or dapsone from discoloration due to oxidation. An alternate antioxidant is BHT.

Any suitable compatible surfactant(s) may be employed in the topical formulations of this invention. Examples of such, surfactants include, but are not limited to ceteareth-20 available as CETOMACROGOL™ 1000, glycerol monostearate, glycerol distearate, glyceryl stearate, polyoxyethylene stearate, a blend of glyceryl stearate and PEG-100 stearate, (as ARLACEL 165), polysorbate 40, polysorbate 60, polysorbate 80, CETETH-20™, sorbitan monopalmitate, sorbitan monostearate, sorbitan monooleate, sorbitan sesquioleate, and mixtures thereof. The amount of surfactant(s) employed in the formulations of this invention will generally be in a w/w % of from about 0.5 to about 1.0%. Generally for the ointment formulations of this invention the amount of surfactants) will generally be from about 0.5 to about 5.0 w/w %, and for the cream formulations of this invention the amount of surfactant(s) will be from about 1.0 to about 10 w/w %.

Any suitable emollient or skin conditioning agent may optionally be included in the topical formulations of this invention. Suitable emollients include, but are not limited to, cholesterol, glycerine, glyceryl monostearate, isopropyl myristate, isopropyl palmitate, cetostearyl alcohol, lanolin alcohols and mixtures thereof. Optionally, dimethicone, mineral oil or white soft paraffin may also be incorporated into the formulations in relatively small amounts to act as a skin conditioner. The emollient or skin conditioning agent may be present in the topical formulations of this invention in a w/w % amount of from about 0.0 to about 40%. In the ointment formulations of this invention the emollient or skin conditioning agent may generally be present in an amount of from about 0.0 to about 10 w/w %, and in the cream or lotion formulations of this invention may generally be present in an amount of from about 2.0 to about 40.0 w/w %.

The formulations of this invention may also optionally contain any suitable penetration enhancer (also called sorption promoters or accelerants). These compositions penetrate into skin to reversibly decrease the barrier resistance. Suitable penetration enhancers include, but are not limited to, diethylene glycol, propylene glycol, ethanol, decanol, monoethyl ether, n-decyl methyl sulfoxide, dimethyl sulfoxide, dimethylacetamide, laurocapram (Azone™), dimethylformamide, sucrose monooleate, and N-methyl-2-pyrrolidine (Pharmasolve™). The penetration enhancer may be present in the formulations of this invention in an amount of from about 0.0 to about 5.0 w/w %. In the ointment formulations of this invention the penetration enhancer may generally be present in an amount of from about 0.0 to about 5 w/w %, and in the cream or lotion formulations of this invention may generally be present in an amount of from about 0.0 to about 5 w/w %.

The formulations of this invention may also optionally include a buffer or neutralizing agent. Examples of suitable buffers include, but are not limited to, citric acid, lactic acid, oleic acid, sodium phosphate, water, triethanolamine, sodium citrate, hydrochloric acid and the like. The buffering agent may be present in the composition in any suitable buffering effective a mount. The gel formulation will generally contain a base, such as for example, sodium hydroxide, triethanolamine and the like. The gel formulations of this invention will also generally include a volatile solvent, such as for example, ethanol, isopropanol and the like.

The formulations of this invention may also include one or more proteases. Proteases are enzymes that conduct proteolysis by hydrolysis of peptide bonds that link amino acids together in polypeptide chains. Proteases are also known as peptidases and proteinases. Preferred proteases contemplated for use in the formulations described herein include protease from Aspergillus, Rhizopus, Bacillus and Serratia sources (Enzymology Research Center, Inc., Miltona, Minn.).

The formulations of this invention may also include a crystallization inhibitor. Crystallization inhibitors are capable of delaying crystallization of the biologically active agent in the delivery vehicle and on the skin. Crystallization inhibitors contemplated for use in the instant invention include, but are not limited to: polyvinylpyrrolidone, polyvinyl alcohols, copolymers of vinyl acetate and vinylpyrrolidone, polyethylene glycols, benzyl alcohol, mannitol, glycerol, sorbitol, polyethoxylated sorbitan esters; lecithin, carboxymethylcellulose sodium, acrylic derivatives such as methacrylates and others, anionic surfactants such as alkali metal stearates, especially of sodium, of potassium ox of ammonium; calcium stearate; triethanolamine stearate; sodium abietate; alkylsulphates, especially sodium laurylsulphate and sodium cetylsulphate; sodium dodecylbenzenesulphonate, sodium dioctylsulphosuccinate; fatty acids, especially those derived from copra oil, canonic surfactants such as water-soluble quaternary ammonium salts of formula N*R′R″R′″R″″, Y′ in which the R radicals are optionally hydroxylated hydrocarbon radicals and Y′ is an anion of a strong acid, such as halide, sulphate and sulphonate anions; cetyltrimethylammonium bromide is among the cationic surfactants utilizable, the amine salts of formula N*R′R″R′″ in which the R radicals are optionally hydroxylated hydrocarbon radicals; octadecylamine hydrochloride is among the cationic surfactants utilizable, the non-ionic surfactants such as optionally polyethoxylated sorbitan esters, in particular Polysorbate 80, polyethoxylated alkyl ethers; polyethylene glycol stearate, polyethoxylated castor oil derivatives, polyglycerol esters, polyethoxylated fatty alcohols, polyethoxylated fatty acids, copolymers of ethylene oxide and propylene oxide, amphoteric surfactants such as substituted lauryl betaine compounds, or preferably a mixture of at least two of these.

In addition, compounds that enhance the activity of cellulase may be employed in the methods of the invention. Such compounds include, but are not limited to, ZM3, a HED2 protein from maize pollen.

The percent by weight of a therapeutic agent of the invention present in a topical formulation will depend on various factors, but generally will be from about 0.01% to about 95% of the total weight of the formulation, and typically about 0.1 to about 25% by weight.

When desired, the herein-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Drops can be delivered via a simple eye dropper-capped bottle, or via a plastic bottle adapted to deliver liquid contents dropwise, via a specially shaped closure.

The compositions described herein may further be formulated for topical administration in the mouth or throat. For example, the compounds may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The compositions of the invention may be applied topically as needed. In some embodiments, the compositions are administered once a day. In other embodiments, the compositions are administered twice a day. In still other embodiments, the compositions are administered every other day. It is contemplated that the compositions of the invention may be administered once a week, twice a week, three times a week, four times a week, five times a week, six times a week, or seven times per week. The frequency and duration of administration can be adjusted, depending on the severity of the individual patient's symptoms.

In further embodiments, the cellulase of the invention may be used prior to, after, or concurrently with other enzymes, including but not limited to, amylases, xylanase, hemicellulase and glucose oxidase.

The compositions and methods described herein are used in the treatment of Morgellons. Although the diagnosis and etiology of Morgellons is controversial, individuals suffering from this painful and debilitating disease experience very real symptoms that require treatment. Morgellons patients often spend decades seeking relief from their symptoms, visiting dozens of physicians and attempting numerous treatments and therapies with antibiotics, anti-fungals, anti-virals, traditional Chinese medicines, herbal and vitamin supplements, topical steroids, NSAIDs, plant extracts, diet modifications, psychotherapy and other treatments. Overwhelmingly, these treatments fail to provide significant relief to Morgellons sufferers. The invention described herein provides treatments and compositions for relief of Morgellons symptoms, whatever the cause, as evidenced by the following testimonials.

The presently disclosed subject matter may be illustrated by the following, non-limiting examples.

### EXAMPLE 1

A 32 ounce spray bottle was filled with 31 ounces water. ⅓ cup (approximately 40 grams) solid particulate Cellulase (Columbus Chemical Industries, Columbus, Wis.; Cat. No. 129600; CAS No. 9012-54-8) was added to the bottle to make a solution of approximately 4%. The bottle was shaken to dissolve the cellulase. The spray nozzle on the bottle was set to mist. The entire body of a Morgellons sufferer was sprayed, saturating all skin, hair and scalp. The face was sprayed with eyes closed to ensure application of the formulation to eyelashes. The formulation was then massaged into the skin by hand. The skin was allowed to air dry (a hair dryer was occasionally used to hasten drying). This treatment was carried out twice daily for three months. The individual reported significant improvements in symptoms.

### EXAMPLE 2

One cup of a powdered composition containing a mixture of 40% cellulase, 20% hemicellulase, 20% xylanase and 20% glucose oxidase (all provided by Columbus Chemical Industries, Columbus, Wis.) was added to a tub full of warm water and swirled to dissolve. A Morgellons patient immersed his entire body up to the neck for 20 minutes, once or twice daily, for a period of two months. The individual reported significant improvements in symptoms.

### EXAMPLE 3

Solid particulate cellulase (Columbus Chemical Industries, Columbus, Wis.; Cat. No. 129600; CAS No. 9012-54-8) was massaged directly into Morgellons lesions and onto the scalp, ankles and feet of a Morgellons sufferer for a period of two months. The individual reported significant improvements in symptoms.

### EXAMPLE 4

An adult Morgellons patient soaked in a tub containing a solution of cellulase (Columbus Chemical Industries, Columbus, Wis.) once a day for 20 minutes each day, for a time period of 2 months. Within one day, the patient described a reduction in swelling and inflammation of the skin. Within several days of initiating treatment, the patient reported feeling very energized for the first time in years. Within one month, the patient reported a black toenail was noticeably cleared. Within two months, the patient reported that brown spots on hands and arms significantly faded, and the skin on the patient's hands shed small white polyp-appearing bumps after black specks-while fibers emerged from under the fingernails. The patient reported that a large nasal polyp cleared up. Large fibers emerged from the patient's head, underarms and lower legs within two months of initiating treatment, and the patient reported continuing improvements in general health after two months of treatment.

### EXAMPLE 5

An adult Morgellons patient topically applied a solution of cellulase (Columbus Chemical Industries, Columbus, Wis.) for a period of approximately two months. The patient reported that after years of trying various oils, ointments, creams, soaps and shampoos that had failed to relieve symptoms, this product actually provided improvements. The patient reported that the biofilm on the scalp was reduced, hair had begun to show some signs of growth, and lesions on the forehead also disappeared. The patient reported previously having lost one eyebrow due to Morgellons, but the eyebrow grew back after treatment.

### EXAMPLE 6

An adult Morgellons patient reported experiencing two separate infections, first by birdmites in 1988 while carrying a turkey over her shoulder and again in 1997 by a co-worker. The patient initially believed she had a flea infestation; however, over time, she experienced more and more crawling sensations and bites. In 1998 the patient began experiencing up to 70 extremely painful biting sensations a minute. The patient eventually stopped working after attempting to treat her workspace with boric acid, an insect growth regulator and an herbal insect repellent. The patient reported experimenting with chemicals she could find in pharmacies, including benzoyl peroxide.

After using a cellulase solution topically (Columbus Chemical Industries, Columbus, Wis.), the patient reported significant improvements in her symptoms, accompanied by the following side effects: body odor, muscle pain, red eyes, flaking skin, and itching.

### EXAMPLE 7

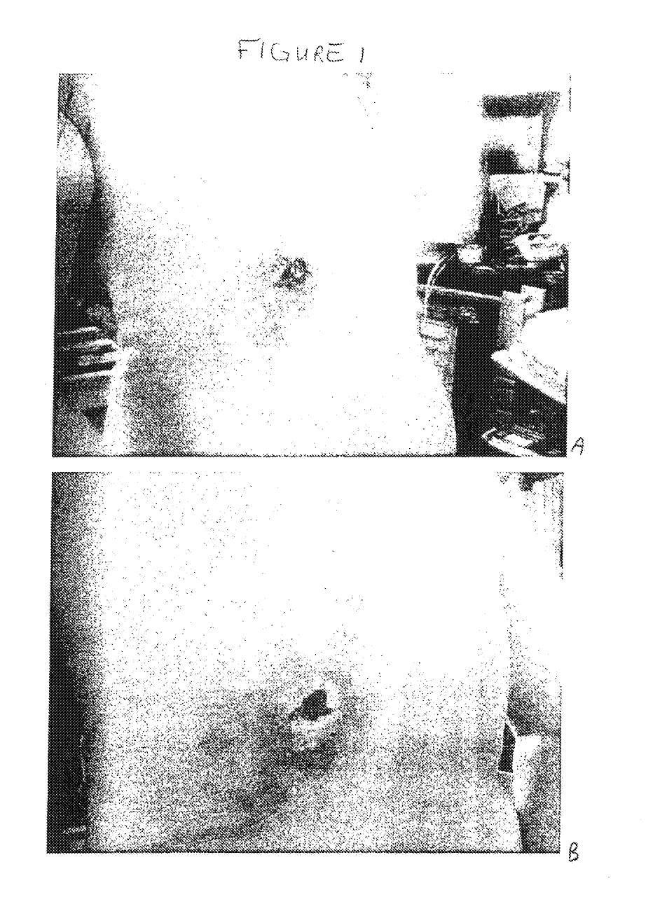

A Morgellons patient had been suffering from a large, deep lesion on the back for several months. The lesion failed to heal using traditional antibiotic therapies (FIG. 1A). A solution of cellulase (Columbus Chemical Industries, Columbus, Wis.) was applied topically and significant improvements were observed within hours of the application (FIG. 1B).

### EXAMPLE 8

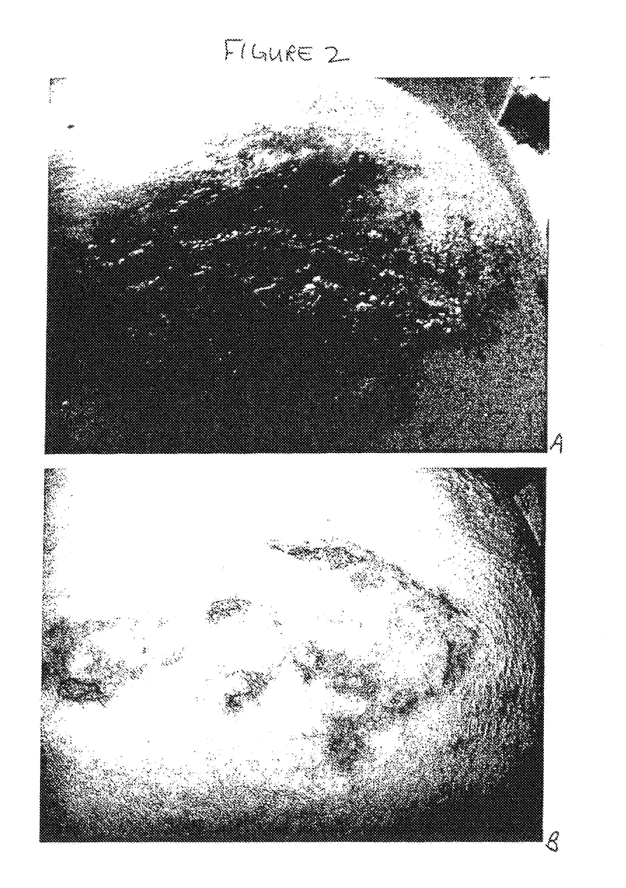

A Morgellons sufferer had a large, open lesion on the skin. The patient applied a solution of cellulase (Columbus Chemical Industries, Columbus, Wis.) topically once or twice daily for 4 months. FIG. 2 shows the lesion (A) before initiating treatment and (B) four months after initiating treatment.

### EXAMPLE 9

A subject suffered from toenail fungus. A pan was filled with several inches of warm water to cover the subject's feet. One tablespoon (approximately 9 grams) of cellulase was added to the water and mixed. Both feet were immersed and soaked for approximately 30 minutes.
The patient reported the emergence of fibers from the cuticles and improvements in the toenails.

### EXAMPLE 10

A second subject suffered from toenail fungus. A solution containing cellulase, hemicellulase, xylanase and glucose oxidase was sprayed on the subject's toenails and scrubbed in with a toothbrush. One of the toenails fell off completely after three soaks at 20 minutes per soak and the tissue under the toenail that had previously been black was restored to a healthy pink.

Solid particulate cellulase (Columbus Chemical Industries, Columbus, Wis.; Cat. No. 129600; CAS No. 9012-54-8) and water are added to a bottle to make a solution of approximately 4%. The bottle is shaken at room temperature to dissolve the cellulase. The spray nozzle on the bottle is set to mist. The entire body of a Morgellons sufferer is sprayed, saturating all skin, hair and scalp. The formulation is then massaged into the skin by hand. The skin is allowed to air dry or dried with a hair dryer to hasten drying. This treatment is carried out once daily for two months.

### EXAMPLE 12

Solid particulate cellulase (Columbus Chemical Industries, Columbus, Wis.; Cat. No. 129600; CAS No. 9012-54-8) and water are added to a bottle to make a solution of approximately 4%. The bottle is shaken at room temperature to dissolve the cellulase. The spray nozzle on the bottle is set to mist. The solution is applied to the feet of an athlete's foot sufferer once or twice dally. The formulation is then massaged into the skin by hand. The skin is allowed to air dry.

### EXAMPLE 13

The feet of an athlete's foot sufferer are immersed in a solution of cellulase for approximately 20 minutes once or twice daily for relief of athlete's foot symptoms.

### EXAMPLE 14

A powdered composition containing 40% cellulase, 20% hemicellulase, 20% xylanase and 20% glucose oxidase is dissolved in water and applied topically to the skin of a patient for relief of Morgellons, onychomycosis or athlete's foot.

### EXAMPLE 15

Equal parts of the following five powdered ingredients are mixed together:

1. Cellulase AN 150,000 CU/GM

2. Amylase 125,000 DU/GM

3. Protease 500,000 HUT/GM

4. Serratiopeptidase 80,000 SPU

5. EDTA (Ethylenediaminetetraacetic Acid) 372.24 F.W.

Components 1-4 are commercially available from Enzymology Research Center, Inc., Miltona, Min. EDTA is available from Columbus Chemical Industries, Columbus, Wis. The above mixture is then employed as described m Examples 1-14 above.

What is claimed is:

1. A topical composition for treatment of Morgellons consisting essentially of a treatment-effective amount of cellulase and a dermatologically acceptable carrier to effectively treat Morgellons upon topical application to skin.

2. The topical composition of claim 1 further comprising an additive selected from the group consisting of a preservative, an antioxidant, a fragrance, a skin penetration enhancer, a colorant, and a sunscreen.

3. The topical composition of claim 1 wherein the topical composition is a cream.

4. The topical composition of claim 1 wherein the topical composition is a lotion.

5. The topical composition of claim 1 wherein the topical composition is a solution.

6. The topical composition of claim 1 wherein the topical composition is an ointment.

7. The topical composition of claim 1 wherein the topical composition is a spray.

8. The topical composition of claim 1, further comprising an additive selected from the group consisting of a preservative, an antioxidant, a fragrance, a colorant, and a sunscreen.

9. The topical composition of claim 1 wherein the topical composition comprises about 3% cellulase.

10. The topical composition of claim 1 wherein the topical composition comprises about 5% cellulase.

11. A composition suitable for topical application, the composition comprising cellulase and a pharmaceutically acceptable carrier.

12. A method for treating Morgellons in a patient, comprising topically applying a dermatological composition to the patient, wherein the dermatological composition comprises an amount of cellulase effective to treat the Morgellons.

13. The method of claim 12 wherein the dermatological composition further comprises an additive selected from the group consisting, of a preservative, an antioxidant, a fragrance, a skin penetration enhancer, a colorant, and a sunscreen.

14. The method of claim 12 wherein the topical composition is a cream.

15. The method of claim 12 wherein the topical composition is a lotion.

16. The method of claim 12 wherein, the topical, composition is a solution.

17. The method of claim 12 wherein the topical composition is an ointment

18. The method of claim 12 wherein the topical composition is a spray.

19. The method of claim 12 wherein the topical composition further comprises an additive selected from the group consisting of a preservative, an antioxidant, a fragrance, a colorant, and a sunscreen.

20. The method of claim 12 wherein the topical composition comprises about 3% cellulase.

21. The method of claim 12 wherein the topical composition comprises about 4% cellulase.

22. The method of claim 1 or claim 12 wherein the topical composition comprises about 7% cellulase.

23. A method for treating onychomycosis or athlete's toot in a patient, comprising topically applying a dermatological composition to the patient, wherein the dermatological composition comprises an amount of cellulase effective to treat the onychomycosis or athlete's foot.

24. The method of claim 23 wherein the dermatological composition further comprises an additive selected from the group consisting of a preservative, an antioxidant, a fragrance, a skin penetration enhancer, a colorant, and a sunscreen,

25. The method of claim 23 wherein the topical composition is a cream.

26. The method of claim 23 wherein the topical composition is a lotion.

27. The method of claim 23 wherein the topical composition is a solution.

28. The method of claim 23 wherein the topical composition is an ointment.

29. The method of claim 23 wherein the topical composition is a spray.

30. The method of claim 23 wherein the topical composition further comprises an additive selected from the group consisting of a preservative, an antioxidant, a fragrance, a colorant, and a sunscreen.

31. The method of claim 23 wherein the topical composition comprises about 3% cellulase.

32. The method of claim 23 wherein the topical composition comprises about 4% cellulase.

33. The method of claim 23 wherein the topical composition comprises about 7% cellulase.

34. A method for treating a skin disorder in a patient, comprising topically applying a dermatological composition to the patient, wherein the dermatological composition comprises art amount of cellulase effective to treat the skin disorder.
